(19) United States
(12) Patent Application Publication
(10) Pub. No.: US 2016/0015264 A1
(43) Pub. Date: Jan. 21, 2016

(54) IMAGING SYSTEM AND METHOD FOR DIAGNOSTIC IMAGING

(71) Applicant: Samsung Electronics Co., Ltd., Suwon-si (KR)

(72) Inventors: Praveen PANKAJAKSHAN, Bangalore (IN); Sandipan CHAKROBORTY, Bangalore (IN)

(73) Assignee: Samsung Electronics Co., Ltd., Suwon-si (KR)

(21) Appl. No.: 14/802,160

(22) Filed: Jul. 17, 2015

(30) Foreign Application Priority Data
Jul. 17, 2014 (IN) 3509/CHE/2014
May 27, 2015 (KR) 10-2015-0073810

(51) Int. Cl. A61B 3/00; A61B 3/14; G03H 1/08; G03H 1/26; G03H 1/16; G06T 7/00; G03H 1/00

(52) U.S. Cl. CPC A61B 3/0025; G06T 7/0014; A61B 3/14; G03H 1/0005; G03H 1/26; G03H 1/16; G03H 1/0808; G06T 2207/30041; G06T 2207/20056; G03H 2001/0816

(57) ABSTRACT

An imaging system and method for using an optical device with an electronic device for diagnostic imaging is provided. The imaging system may include a controller configured to capture a series of holograms by powering a light source of the optical device to illuminate an object, wherein light from the light source is collimated onto the object through an aperture of the optical device. The controller may be configured to extract an interference pattern of the object from the series of holograms, wherein the interference pattern is produced by interference between a reflected beam from the object and a reference beam formed by a diffraction mirror of the optical device. The controller may be configured to record at least one image of the object based on the interference pattern. The imaging system may include a data storage configured to store the at least one image.

### CROSS-REFERENCE TO RELATED APPLICATION(S)

This application claims the benefit under 35 USC 119(a) of Indian Patent Application No. 3509/CHE/2014, filed on Jul. 17, 2014, in the Indian Patent Office, and Korean Patent Application No. 10-2015-0073810, filed on May 27, 2015, in the Korean Intellectual Property Office, the entire disclosures of which are incorporated herein by reference for all purposes.

### BACKGROUND

1. Field

The following description relates to a healthcare system, and more particularly, to an imaging system for recording an image of an eye of a user.

2. Description of Related Art

In general, some hand-held optical adaptors include a function of capturing an image of a user's anatomy, for example, the skin, an eye, and an ear. A portion of the hand-held optical adaptors includes an interchangeable instrument available for a variety of medical examinations to capture an image. Some optical adaptors are designed to be used with an imaging capturing device having camera features and functions. An optical adaptor may be attached to an imaging capturing device by an outer housing facility of the optical adaptor on a side of the optical adaptor on which an eye of a user may be placed for examination.

In rural areas, many persons suffer from infections of, for example, an eye, an ear, and skin. Infections mainly in the eye, such as cataracts, may be cured or prevented if they are detected early. Due to an absence of expensive optical adaptors and a lack of experts in rural areas, it is difficult to detect such infections early in the rural areas. However, an innovative imaging system including an optical adaptor that is attached to an electronic device captures images of an affected eye of a person using differential transmission holography, optical fluorescence, or an array of lenses capturing reflected light. An optical adaptor attached to a smartphone having a camera lens and a display system captures a low-resolution image since an optical resolution of the camera lens is low.

Captured images are sent to a location remotely located from a user, such as a hospital/laboratory, over an existing wireless network at which experts use the images for diagnosis and provide the user with necessary preventive measures. The above procedure consumes a relatively large amount of time since the images are sent to the remote location for diagnosis, and also has an increased standby time until the images are used by the experts. A hand-held processing device such as a phone or a remote server may be selected as a processing unit based on image resolution and complexity.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, there is provided an imaging system for using an optical device with an electronic device for diagnostic imaging. The imaging system may include a controller configured to capture a series of holograms by powering a light source of the optical device to illuminate an object, wherein light from the light source is collimated onto the object through an aperture of the optical device. The controller may be configured to extract an interference pattern of the object from the series of holograms, wherein the interference pattern is produced by interference between a reflected beam from the object and a reference beam formed by a diffraction mirror of the optical device. The controller may be further configured to record at least one image of the object based on the interference pattern. The imaging system may include a data storage configured to store the at least one image.

When recording the at least one image of the object based on the interference pattern, the imaging system may be configured to: obtain a frequency spectrum of the object by obtaining a Fresnel transform of an amplitude and a phase retrieved from the interference pattern, wherein a high frequency portion in the frequency spectrum is recovered using an iterative restoration approach; and obtain the at least one image of the object by obtaining a Fourier transform of the frequency spectrum, wherein the at least one image is a low-resolution image.

When recording the at least one image of the image based on the interference pattern, the imaging system may be configured to: obtain a frequency spectrum of the object by obtaining a Fresnel transform of an amplitude and a phase retrieved from the interference pattern, wherein a high frequency portion in the frequency spectrum is recovered using an iterative restoration approach; and obtain the at least one image of the object by obtaining an inverse Fourier transform of the frequency spectrum, wherein the at least one image is a high-resolution image.

The light may be partially reflected and partially transmitted by a beam splitter.

The light may be split by the beam splitter into an incident beam and the reference beam, and the incident beam may pass through a phase plate and be reflected from the object.

The reference beam may be formed by the light source.

The optical device may include an adaptor. The adaptor may include a housing facility including a proximal end and a distal end, the housing facility being configured to removably attach to the electronic device at the proximal end. The proximal end may be configured to surround an imaging sensor of the electronic device and the distal end is configured to be fixed on or near the object using a head strap.

The imaging system may be further configured to display the recorded at least one image on the electronic device and authenticate the recorded at least one image by comparing the recorded at least one image to at least one pre-stored image of the object.

In another general aspect, there is provided a method of operating an optical device. The method may include capturing a series of holograms by powering a light source associated with the optical device to illuminate an object, wherein light from the light source is collimated onto the object through an aperture. The method may include extracting an interference pattern of the object from the series of holograms, wherein the interference pattern is produced by interference between a reflected beam from the object and a reference beam formed by a diffraction mirror associated with the optical device. The method may further include recording at least one image of the object based on the interference pattern, and storing the at least one image in a data storage of an electronic device.

The recording of the at least one image may include: obtaining a frequency spectrum of the object by obtaining a Fresnel transform of an amplitude and a phase retrieved from the interference pattern, wherein a high frequency portion in the frequency spectrum is recovered using an iterative restoration approach; and obtaining the at least one image of the object by obtaining a Fourier transform of the frequency spectrum, wherein the at least one image is a low-resolution image.

The recording of the at least one image may include: obtaining a frequency spectrum of the object by obtaining a Fresnel transform of an amplitude and a phase retrieved from the interference pattern, wherein a high frequency portion in the frequency spectrum is recovered using an iterative restoration approach; and obtaining the at least one image of the object by obtaining an inverse Fourier transform of the frequency spectrum, wherein the at least one image is a high-resolution image.

The light may be partially reflected and partially transmitted by a beam splitter.

The light may be split by the beam splitter into an incident beam and the reference beam, and the incident beam may pass through a phase plate and be reflected from the object.

The reference beam may be formed by the light source.

The method may include displaying the recorded at least one image on the electronic device and authenticating the recorded at least one image by comparing the recorded at least one image to at least one pre-stored image of the object.

In another general aspect, an imaging system for recording at least one image of an object includes a housing facility including a light source, an aperture, a diffraction mirror, a head strap, a display screen, a data storage, and a controller. The housing facility may include a proximal end and a distal end, and may be configured to attach to the display screen at the proximal end. The controller may be configured to capture a series of holograms by powering the light source to illuminate the object, wherein light from the light source is collimated onto the object through the aperture. The controller may be configured to extract an interference pattern of the object from the series of holograms, wherein the interference pattern is produced by interference between a reflected beam from the object and a reference beam formed by the diffraction mirror. The controller may be configured to record at least one image of the object based on the interference pattern, and store the at least one image in the data storage.

When recording the at least one image of the object based on the interference pattern, the controller may be further configured to: obtain a frequency spectrum of the object by obtaining a Fresnel transform of an amplitude and a phase retrieved from the interference pattern, wherein a high frequency portion in the frequency spectrum is recovered using an iterative restoration approach; and obtain the at least one image of the object by obtaining a Fourier transform of the frequency spectrum, wherein the at least one image is a low-resolution image.

When recording the at least one image of the object based on the interference pattern, the controller may be further configured to: obtain a frequency spectrum of the object by obtaining a Fresnel transform of an amplitude and a phase retrieved from the interference pattern, wherein a high frequency portion in the frequency spectrum is recovered using an iterative restoration approach; and obtain the at least one image of the object by obtaining an inverse Fourier transform of the frequency spectrum, wherein the at least one image is a high-resolution image.

The light may be partially reflected and partially transmitted by a beam splitter.

The controller may be further configured to display the recorded at least one image on the display screen and authenticate the recorded at least one image by comparing the recorded at least one image to at least one pre-stored image of the object.

In yet another general aspect, an imaging adaptor may include a housing configured to attach to an image sensor of an electronic device, and configured to be fixed to or near an object. The imaging adaptor may be configured to emit light towards the object, capture a series of holograms generated by light reflected from the object, and generate an interference pattern from the series of holograms. The interference pattern may be configured to be processed to record at least one image of the object.

The electronic device may be a smartphone.

The object may be an eye.

Other features and aspects will be apparent from the following detailed description, the drawings, and the claims.

### BRIEF DESCRIPTION OF THE DRAWINGS

FIG. 1 is a diagram illustrating a system for using an optical adaptor with an electronic device to record an image of an object of a user, according to an embodiment.

FIG. 2 is a diagram illustrating a system including various components in an optical adaptor of which one end is attached to an electronic device and another end is fixed to an object, according to an embodiment.

FIG. 3 is a block diagram illustrating components included in an electronic device or a server, according to an embodiment.

FIG. 4 is a perspective view illustrating an imaging system including an optical adaptor attached to an electronic device, according to an embodiment.

FIG. 5 is a diagram illustrating an operation of components included in an optical adaptor, according to an embodiment.

FIG. 6 is a diagram illustrating a process of reconstructing a low-resolution image in an electronic device, according to an embodiment.

FIG. 7 is a diagram illustrating a process of reconstructing a high-resolution image in a server, according to an embodiment.

FIG. 8 is a graph showing a waveform representing a relationship between light transmittance and a wavelength, according to an embodiment.

FIGS. 9A and 9B illustrate examples of a retinal dimension of an eye and a size of a donut-shaped illumination, according to an embodiment.

FIG. 10 is a flowchart illustrating a method of using an optical adaptor with an electronic device to record an image of an object of a user, according to an embodiment.

Throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent to one of ordinary skill in the art. The sequences of operations described herein are merely examples, and are not limited to those set forth herein, but may be changed as will be apparent to one of ordinary skill in the art, with the exception of operations necessarily occurring in a certain order. Also, descriptions of functions and constructions that are well known to one of ordinary skill in the art may be omitted for increased clarity and conciseness.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

The examples herein and the various features and advantageous details thereof are explained more fully with reference to the non-limiting examples that are illustrated in the accompanying drawings and detailed in the following description. Descriptions of well-known components and processing techniques are omitted so as to not unnecessarily obscure the examples herein. Also, the various examples described herein are not necessarily mutually exclusive, as some examples can be combined with one or more other examples to form new examples. The term “or” as used herein, refers to a non-exclusive or, unless otherwise indicated. The examples used herein are intended merely to facilitate an understanding of ways in which the examples herein can be practiced and to further enable those skilled in the art to practice the examples herein. Accordingly, the examples should not be construed as limiting the scope of the examples herein.

The examples herein disclose an imaging system and method for recording an image of an object. The imaging system includes a housing facility, an imaging sensor, a light source configured to make light partially coherent, a phase plate configured to generate a donut-shaped illumination, a beam-splitter cube configured to partially reflect and transmit the light, a diffraction mirror configured to form a reference beam, a head strap configured to fix the optical adapter on the object, and a rechargeable battery pack. The housing facility is attached to the display screen at a proximal end and extends from the proximal end to a distal end.

The method includes powering the light source to emit the light toward the object. The light from the light source is collimated onto the object through a pinhole aperture. Further, the method includes extracting an interference pattern of the object based on the emitted light. The interference pattern is obtained by interference between a reflected beam from the object and the reference beam. Further, the method includes obtaining a frequency spectrum of the object by obtaining a Fresnel transform of an amplitude and a phase retrieved from the interference pattern. A high frequency portion in the frequency spectrum is recovered using an iterative restoration approach. Further, the method includes obtaining the image of the object by obtaining a Fourier transform of the frequency spectrum. Further, the method includes recording the image in the imaging system for diagnosis.

The method and system disclosed herein is simple and robust for building a low-cost, hand-held optical adapter capable of imaging an eye, an ear, or a throat noninvasively, and diagnosing conditions. The optical adapter also reads microscopic information for verification of its authenticity. The optical adapter fitting includes the housing facility that is attachable to the electronic device and the housing facility contains a light transmission guide configured to focus the light from a partially coherent light emitting diode (LED) light source and to direct the light onto the object being viewed. The light transmission guide includes several components, such as a pinhole aperture configured to collimate light from the light source to make the light partially coherent, a beam-splitter cube configured to partially reflect and transmit the light, a diffraction mirror configured to form a reference beam, and a phase plate configured to generate a donut-shaped illumination.

The imaging system disclosed herein is a low-cost and hand-held optical adapter for imaging an eye, an ear, or a throat, and diagnoses existing or developing conditions using a consumer camera. Using iterative methods, a high-resolution image is reconstructed from a relatively low-resolution image and an optical adapter to capture a wide angle scene over a narrow angle lens system is designed. Captured images are of low cost but comparable in image quality to expensive diagnostic equipment. The object is imaged noninvasively and no ionizing radiation is used. Also, the proposed method and system may be implemented using existing optical components and does not require extensive setup and instrumentation.

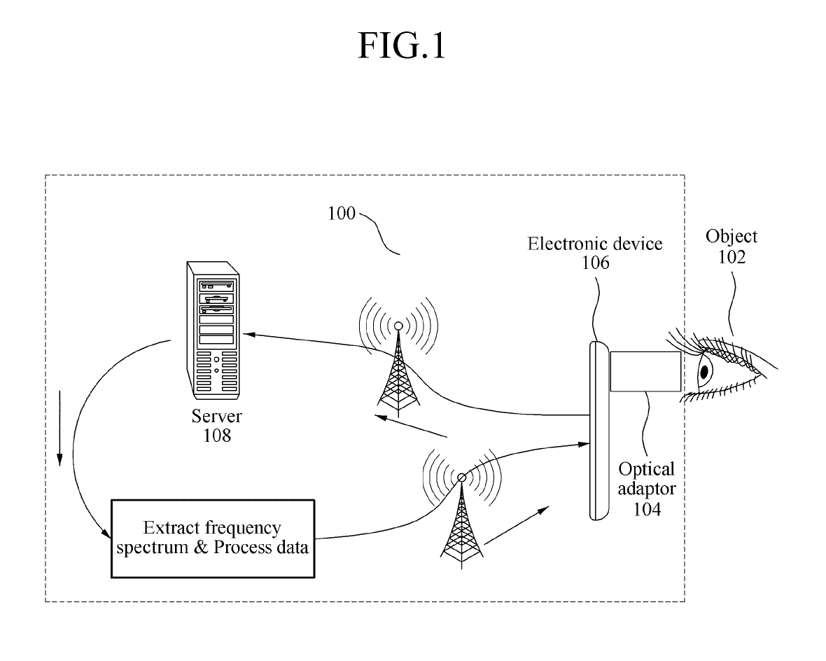

Hereinafter, examples will be described with reference to FIGS. 1 through 10.

FIG. 1 illustrates an example of a system 100 for using an optical adaptor with an electronic device to record an image of an object of a user. Referring to FIG. 1, the system 100 includes an optical adaptor 104, an electronic device 106, and a server 108. The optical adaptor 104 is provided to an object 102.

In an example, the object 102 refers to, for example, an eye, an ear, a throat, skin, currency, or a document. However, the object 102 is not limited to the aforementioned examples. The object 102 is positioned on the optical adaptor 104 to noninvasively image a subject of the object 102 for the purpose of diagnosing and evaluating the object 102. For example, the optical adaptor 104 may be fixed to an eye corresponding to the object 102 to noninvasively image a retina, for example, the subject, of the eye in order to diagnose and evaluate the eye.

In an example, the optical adaptor 104 may be attached to the electronic device 106 to perform many examinations that are currently performed by standard ophthalmoscopes in order to view the retina of the user, and captures images of the retina of the user.

In an example, the optical adaptor 104 is attached to the electronic device 106 through a snap-fit connection, a sliding connection, or other mechanisms for fixing the optical adaptor 104 to the electronic device 106. The optical adaptor 104 may be removably attached to the electronic device 106, allowing the optical adaptor 104 to be attached when an optical system is in use, and detached when the optical system is not in use. Other types of fixed and removable attachment methods and mechanisms may be used to fix the optical adaptor 104 to the electronic device 106, in addition to the examples provided herein. The optical adaptor 104 is removably attached to the electronic device 106 at a proximal end of the optical adapter 104 and extends from the proximal end to a distal end of the optical adapter 104. The proximal end of the optical adaptor 104 surrounds an imaging sensor in the electronic device 106. The distal end of the optical adaptor 104 is fixed to or positioned on the object 102 to be imaged, diagnosed, and evaluated. The optical adaptor 104 emits the light toward the object 102 to generate an interference pattern of the object 102. The captured image is received by the electronic device 106 using the imaging sensor.

In an example, the electronic device 106 described herein may be, without being limited, for example, a laptop, a desktop computer, a mobile phone, a smartphone, a personal digital assistant (PDA), a tablet, a phablet, a consumer electronic device, or other electronic devices.

The electronic device 106 is attached to the optical adaptor 104. The electronic device 106 may be configured to take a photo of an interference pattern captured at a focus of the imaging sensor. The interference pattern is generated by the optical adaptor 104 by emitting the light toward the object 102. The electronic device 106 may be configured to obtain a frequency spectrum of the object 102 by obtaining a Fresnel transform of an amplitude and a phase retrieved from the interference pattern. The electronic device 106 may be configured to obtain an image of the object 102 by obtaining a Fourier transform of the frequency spectrum.

In an example, extracting and processing of the interference pattern may be performed by the electronic device 106 to reconstruct a low-resolution image. In another example, to reconstruct a high-resolution image, the electronic device 106 may be configured to transmit the captured interference pattern to the server 108 in order for the server 108 to extract and process spectrum data. The electronic device 102 includes an interface suitable for directly or indirectly communicating with the server 108 and other various devices.

In an example, the server 108 described herein may be, without being limited, for example, a gateway device, a router, a hub, a computer, or a laptop. The server 108 may be configured to receive the interference pattern from the electronic device 106. The server 108 may be configured to extract the frequency spectrum of the object 102 obtained by the Fresnel transform of the amplitude and the phase retrieved from the interference pattern to record an image of the object 102 in the server 108. The server 108 may be configured to transmit the processed and reconstructed high-resolution image to the electronic device 106 in order to display the reconstructed image and thereby diagnose existing or developing conditions.

Conventional systems may not perform noninvasive imaging of an eye/ear without ionizing radiations since an optical resolution of an integrated consumer mobile camera is low and is inapplicable to medical application fields. Dissimilar to the conventional systems, an optical adaptor and an electronic device combined with a backend computation operation replace an expensive high-resolution lens system without moving parts and using a lensless holography method for computationally reconstructing an image from a light interference pattern.

FIG. 1 illustrates a limited overview of the system 100, however, it should be understood that another example is not limited thereto. Also, the system 100 may include different components or modules mutually communicating with other hardware or software components. For example, reconstruction of the low-resolution image is performed by the electronic device 106. In an example, reconstruction of the high-resolution image is performed by the server 108.

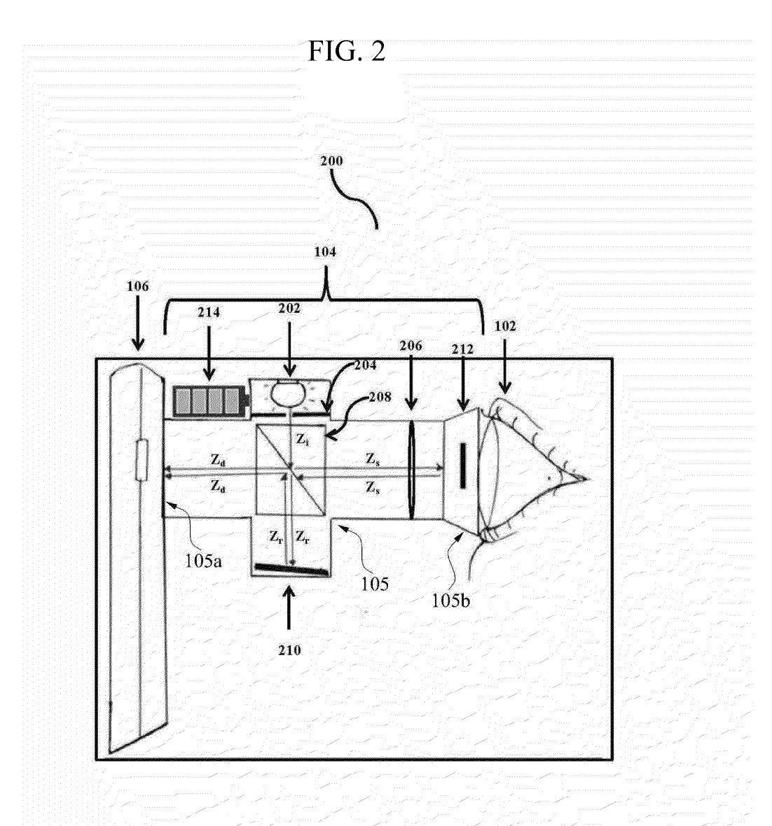

FIG. 2 illustrates an example of a system 200 including various components in an optical adapter 104 of which one end is attached to an electronic device 106 and another end is fixed to an object 102. In an example, the optical adapter 104 includes a housing facility 105, a light source 202, a pinhole aperture 204, a phase plate 206, a beam-splitter cube 208, a diffraction mirror 210, a head strap 212, and a rechargeable battery pack 214.

The housing facility 105 is removably attached to the electronic device 106 at a proximal end and extends from the proximal end 105a to a distal end 105b. The head strap 212 is provided at the distal end to fix the optical adapter 104 on the object 102.

The light source 202 emits light to illuminate the object 102. In an example, the light source 202 may be an LED or a light amplification by stimulated emission of radiation (LASER). For example, an LED system may provide adequate brightness and intensity to effectively illuminate the object 102 of the user if focused properly. The light source 202 may be configured to direct the light only to an interior side of the optical adapter housing facility 105. The rechargeable battery pack 214 is used in association with the light source 202 to power the light source 202. The pinhole aperture 204 may be configured to collimate the light from the light source 202 to make the light partially coherent.

The phase plate 206 generates a donut-shaped illumination. The beam-splitter cube 208 partially reflects and transmits the light emitted from the light source 202. Herein, that light is partially reflected and partially transmitted, or vice versa, indicates that a portion of light is reflected and a portion of light is transmitted.

Further, the partially coherent light is split by the beam-splitter cube 208 into an incident beam and a reference beam. The incident beam is partially transmitted and partially reflected by the beam-splitter cube 208. The incident beam passes through the phase plate 206 and then is reflected from the object 102. The diffraction mirror 210 reflects the incident beam partially reflected by the beam-splitter cube 208. The rechargeable battery pack 214 is used in association with the light source 202 to power the light source 202. The partially coherent light is reflected back from the object 102 to the imaging sensor of the electronic device 106.

Notations of FIG. 2 are defined as follows:

Zₗ denotes a distance between the light source 202 and a center of the beam-splitter cube 208.

Zᵣ denotes a distance between the center of the beam-splitter cube 208 and the diffraction mirror 210.

Zₛ denotes a distance between a specimen, or object, and the center of the beam-splitter cube 208.

Z_{d} denotes a distance between the imaging sensor and the center of the beam-splitter cube 208.

Based on the above notations, distances traversed by the reference beam and the reflected beam are calculated as follows:

Distance traversed by the reference beam=Zᵢ+2Zᵣ+Z_{d}

Distance traversed by the reflected beam=Zᵢ+2Zₛ+Z_{d}

The electronic device 106 captures and processes an image of the object 102 by extracting the interference pattern. An example operation of the electronic device 106 for capturing and processing an image of the object will now be described.

For example, in a scenario in which an ear of a user, such as a patient, is to be imaged, the proximal end 105a of the optical adapter 104 is fixed to a smartphone, surrounding the imaging sensor on the smartphone 106. The distal end 105b of the optical adapter 104 is fixed to the ear of the user through the head strap 212. The LED light source 202 in the optical adapter 104 is activated to emit light beams for illuminating the ear of the user.

The light emitted from the LED light source 202 passes through the pinhole aperture 204 to collimate the light, in order to make the light partially coherent. The partially coherent light is split by the beam-splitter cube 208 into the incident beam and the reference beam. The incident beam is partially transmitted and partially reflected by the beam-splitter cube 208. The incident beam passes through the phase plate 206 and is emitted to illuminate the ear of the user. The phase plate 206 generates the donut-shaped illumination to reduce the reflection from the ear. The incident beam is reflected back from the ear to the imaging sensor of the smartphone 106 along with the reference beam reflected by the diffraction mirror 210. The imaging sensor of the smartphone 106 receives an interference pattern of the ear. That is, the incident beam reflected back from the ear interferes with the reference beam reflected back from the diffraction mirror 210.

The smartphone 106 extracts a frequency spectrum of the ear by obtaining a Fresnel transform of an amplitude and a phase recovered from the interference pattern. When the image is a low-resolution image, the smartphone 106 reconstructs the image of the ear by obtaining a Fourier transform of the frequency spectrum. When the image is a high-resolution image, the smartphone 106 transmits the interference pattern to the server 108 to extract and process spectrum data, in order to record the image of the ear.

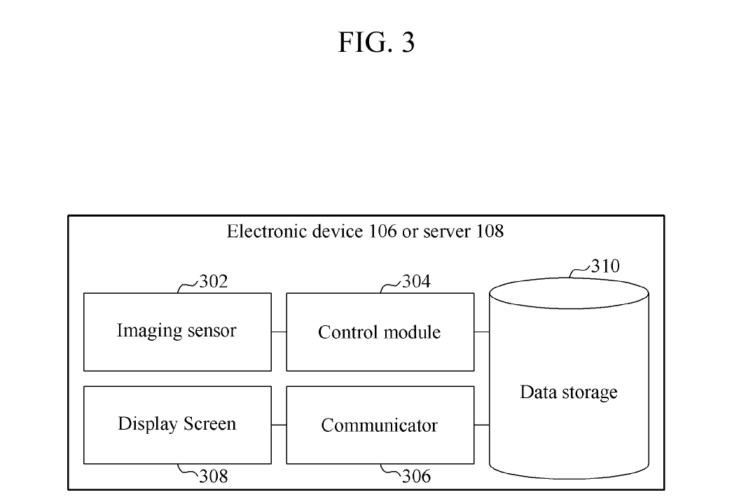

FIG. 3 illustrates an example of components included in an electronic device 106 or a server 108.

Referring to FIG. 3, the electronic device 106 includes an imaging sensor 302, a control module or controller 304, a communication module or communicator 306, a display or display screen 308, and a data storage 310. The imaging sensor 302 is configured to capture a series of holograms that are partially reflected from an object.

In an example, the imaging sensor 302 described herein may be, without being limited, for example, a charge-coupled device (CCD) imaging sensor and a complementary metal-oxide-semiconductor (CMOS) imaging sensor.

The controller 304 is configured to extract an interference pattern of an object from a series of holograms. The interference pattern is obtained by interference between a reflected beam from the object and a reference beam from a diffraction mirror. The controller 304 may be configured to extract the interference pattern prior to determining a calibration factor in the electronic device 106 by the imaging sensor 302 in a housing facility. The controller 304 may be configured to obtain a frequency spectrum of the object by obtaining a Fresnel transform of an amplitude and a phase retrieved from the interference pattern. A high frequency portion in the frequency spectrum may be recovered using an iterative restoration approach. The controller 304 may be configured to obtain an image of the object by obtaining a Fourier transform of the frequency spectrum.

In an example, the image may be a low-resolution image. The controller 304 may be configured to record the image of the object in the data storage 310. The controller 304 may include, for example, a visual dimension system.

The communicator 306 may be configured to transfer captured data to the server 108 in order for the server 108 to extract the frequency spectrum of the interference pattern and process the interference pattern in order to reconstruct the image of the object. Further, the display screen 308 may be configured to display the reconstructed image to diagnose existing or developing conditions. The data storage 310 may be configured to store various images of the object 102. The data storage 310 may be configured to store reconstructed images of the object 102 to diagnose existing or developing conditions. The data storage 310 may be configured to store control instructions to perform various operations in a system.

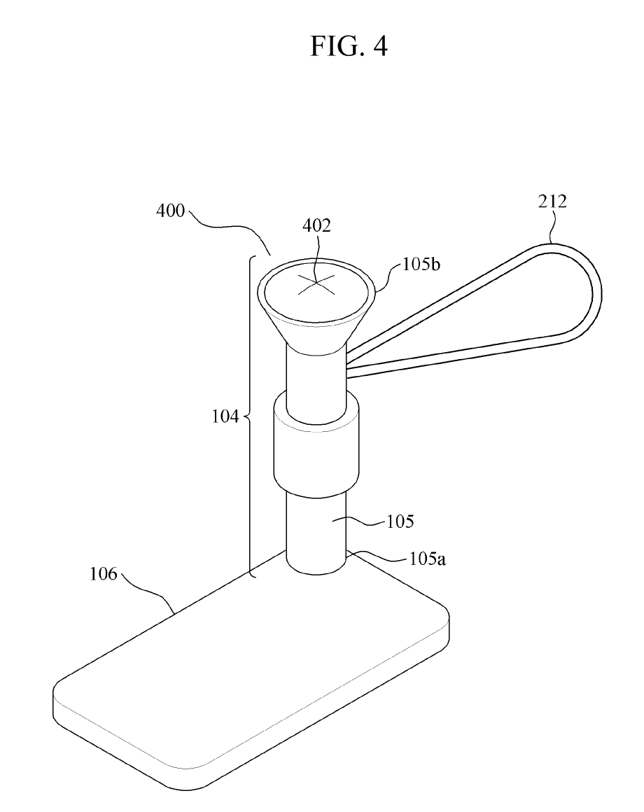

FIG. 4 illustrates an example of an imaging system 400 including an optical adapter 104 attached to an electronic device 106. In this example, the optical adapter 104 is attached to the electronic device 106 at a proximal end 105a through a snap-fit connection, a sliding connection, or other mechanisms for fixing the optical adapter 104 to the electronic device 106, and extends from the proximal end 105a to a distal end 105b. The proximal end 105a of the optical adaptor 104 surrounds an imaging sensor (not shown) on the electronic device 106. A head strap 212 fixes a specimen of a user, for example, a patient. A cross hair 402 refers to a net of fine lines or fibers in the eyepiece of a sighting device for fixing the specimen or an object to the optical adapter 104.

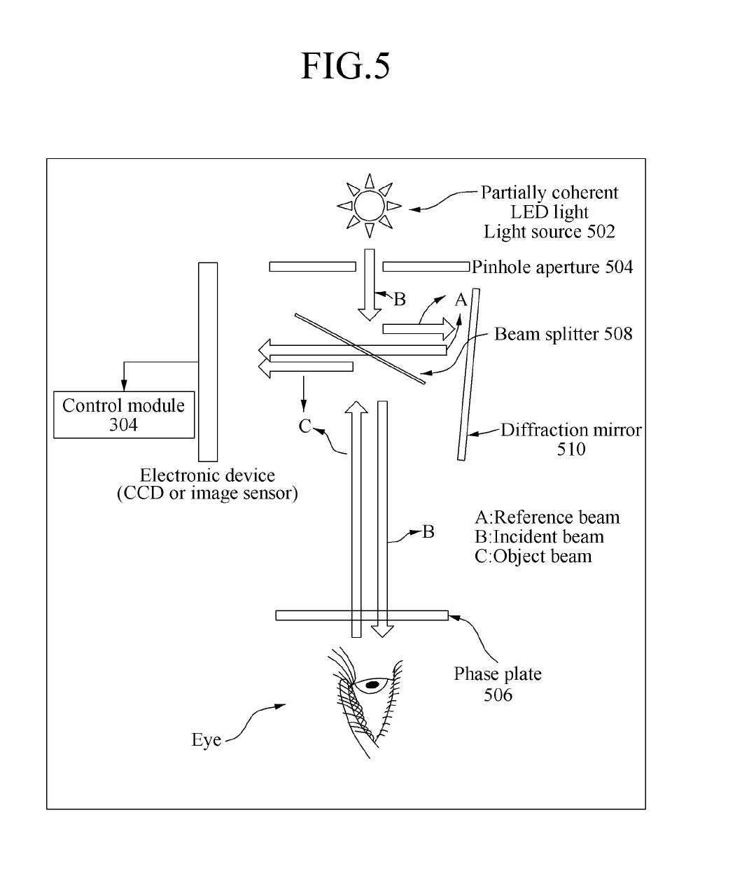

FIG. 5 illustrates an example of an operation of components included in an optical adapter to illuminate an object with partially coherent light. LED light is emitted from a light source 502 to illuminate an eye fixed to a distal end of an optical adapter (not shown). The LED light is directed only to the interior side of an optical adapter housing facility. The LED light passes through a pinhole aperture 504 configured to collimate the light to make the light partially coherent. The partially coherent light passing through the pinhole aperture 504 may be considered as an incident beam emitted from the light source 502 to illuminate the eye, and is marked with a notation “B”.

A beam splitter 508 partially reflects and transmits the partially coherent LED light. The beam splitter 508 splits the partially coherent LED light into an incident beam and a reference beam. The reference beam is marked with a notation “A”. A diffraction mirror 510 reflects the reference beam received from the beam splitter 508. The transmitted incident beam “B” passes through a phase plate 506 and is then emitted toward the eye to be studied. The incident beam “B” passes through the phase plate 506 to generate a donut-shaped illumination, in order to avoid pupil reflections of the eye. An object beam marked with a notation “C” and reflected from the eye or retina interferes with the reflected reference beam “A” from the diffraction mirror 510, thereby generating an interference pattern.

The interference pattern is collected by an imaging sensor (not shown) of an electronic device and transmitted to a controller 304 included in the electronic device or a server (not shown). A frequency spectrum of the object is obtained by a Fresnel transform of an amplitude and a phase of the interference pattern. An image of the object is reconstructed by obtaining a Fourier transform of the frequency spectrum of the object to display the reconstructed image on a display screen (not shown) to diagnose existing or developing conditions.

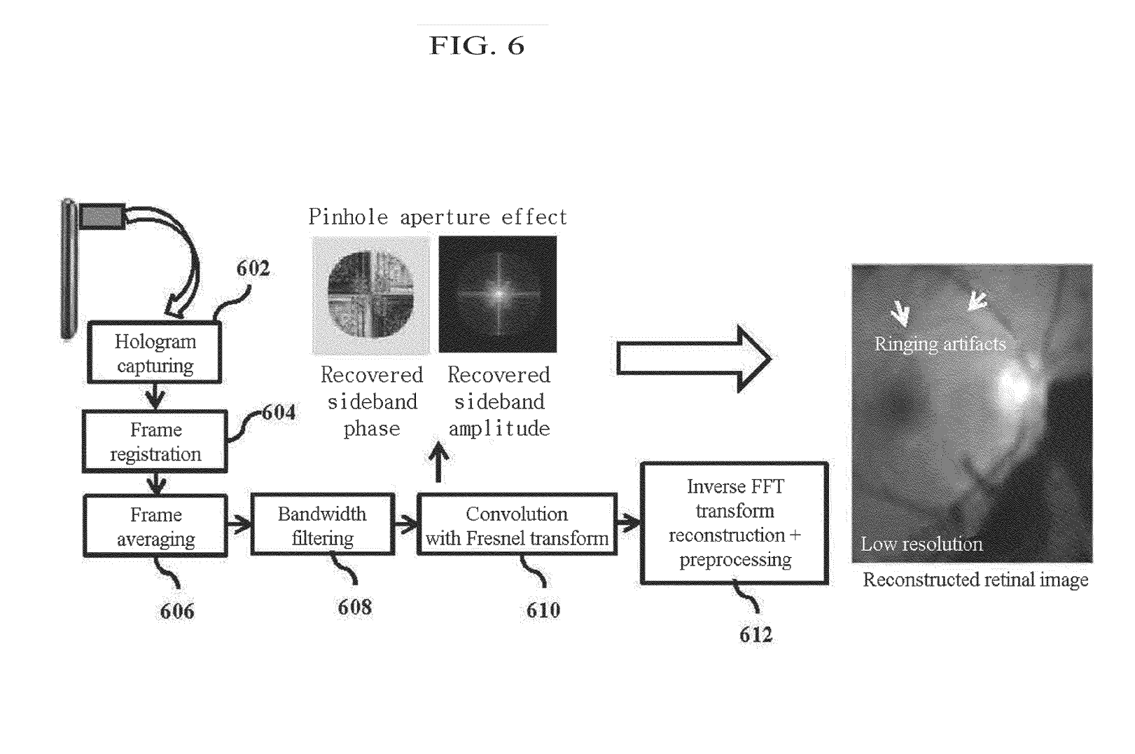

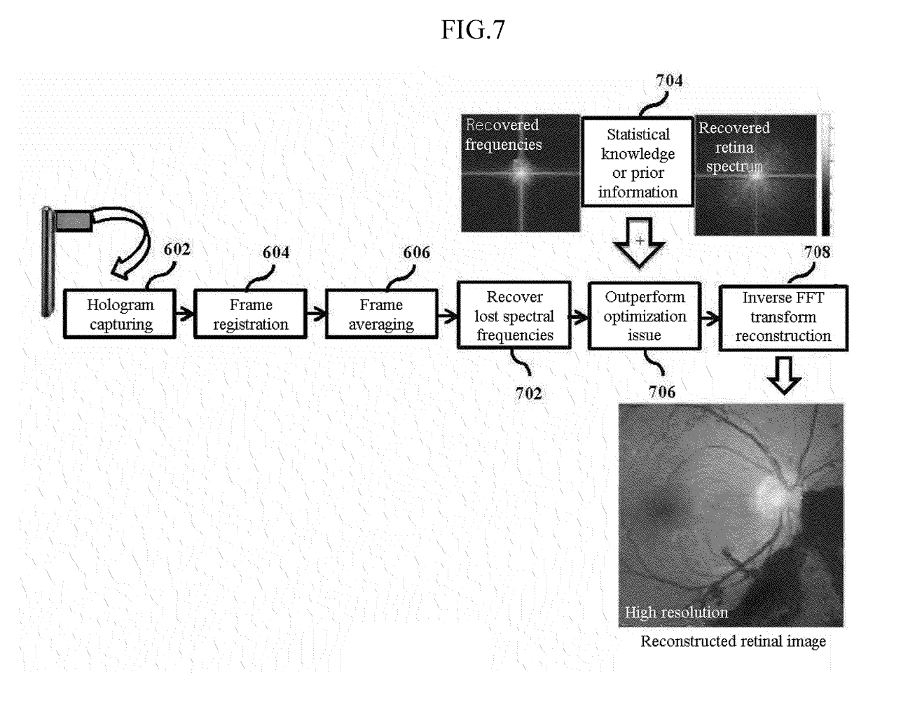

FIG. 6 illustrates an example of a process of reconstructing a low-resolution image in an electronic device. In operation 602, the imaging sensor 302 of FIG. 3 captures images of an object at N frames/sec. In operation 602, the imaging sensor 302 captures eight observed holograms/frames. In operation 604, the eight observed frames are registered. Upon registering the eight observed frames, the average of the eight observed frames is calculated to improve a signal-to-noise ratio (SNR) in operation 606. Upon determining the average of the eight observed frames, a principal energy e(u, v) is extracted. In operation 608, a bandwidth filter filters the principal energy with the defined bandwidth limits to remove a direct current (DC) component and twin images within the eight observed frames. In operation 610, a frequency spectrum of an image is obtained by obtaining a Fresnel transform of an amplitude and a phase recovered from the captured images. In operation 612, a low-resolution image of the object is reconstructed by obtaining an inverse Fourier transform of the frequency spectrum and pre-processed. Further, a high-resolution image of the object is reconstructed by the inverse Fourier transform of the frequency spectrum as shown in the FIG. 7.

FIG. 7 illustrates an example of a process of reconstructing a high-resolution image in a server. The high-resolution image is reconstructed from a relatively low-resolution image using iterative methods. Following operation 606 of FIG. 6, in operation 702, an amplitude and a phase of an image are recovered using an optimization algorithm. In operation 704, statistical prior knowledge is added to iteratively reconstruct the high-resolution image of the object and a high frequency portion from the frequency spectrum of the object. In operation 706, the optimization issue is outperformed by adding the statistical prior knowledge of the image. In operation 708, the high-resolution image of the object is reconstructed by the inverse Fourier transform of the frequency spectrum. The consecutive reconstructed images are registered to correct the motion artifact and a super high-resolution image of the object is obtained from a simple narrow angle less-less system.

Hereinafter, a process of reconstructing a low-resolution image from an interference pattern in an electronic device and a process of reconstructing a high-resolution image in a server will be described.

When an object pattern on a CCD imaging sensor of an electronic device is s(u, v) and a reference beam pattern is r(u, v), an interference pattern e(u, v) between the object pattern and the reference beam pattern is expressed by Equation 1.

e(u,v)=|s(u,v)|²+|r(u,v)|²+s(u,v)r*(u,v)+s*(u,v)r(u,v) [Equation 1]

In Equation 1, the reference beam pattern is given by Equation 2.

$$r ( u , v ) = {r}_{0} exp ( j \frac{2 π}{λ} u sin θ ) \qquad [ Equation 2 ]$$

In Equation 2, r₀ denotes a known constant amplitude, λ denotes a wavelength of light used, and θ denotes an angle of the reference beam, such that θₘₐₓ≈λ/2Δu with sampling Δu. The terms |s(u,v)|² and |r(u,v)|² denote DC terms while s*(u,v)r(u,v) is a twin image. An object complex field s(u, v) is reconstructed from e(u, v) by suppressing the DC terms and the twin image. Equation 3 is obtained using a Bayesian framework, to minimize a cost function.

J(s(u,v)|e(u,v))=½∥e(u,v)−(u,v)−(|s(u,v)|²+|r(u,v)|²+s(u,v)r*(u,v)+s*(u,v)r(u,v))∥₂²+λJ(s(u,v)) [Equation 3]

In Equation-3, the cost function to be minimized is J(s(u,v)|e(u,v)) and prior knowledge on a complex spectrum to be estimated is given by J(s(u,v)). A parameter A used here denotes a tradeoff parameter and is not to be confused with the wavelength of light. The prior knowledge is defined as Equation 4.

J(s(u,v))=∥∇s(u,v)∥₁ [Equation 4]

An iterative solution to estimate s(u, v) is obtained using a simple gradient descent, as expressed by Equation 5.

ŝ(u,v)ⁿ⁺¹=ŝ(u,v)ⁿ⁺¹−α∇J(ŝ(u,v)ⁿ|e(u,v)) [Equation 5]

In Equation 5, ∇J(ŝ(u,v)ⁿ|e(u, v)) denotes a gradient of the cost function when no statistical prior knowledge is introduced. The gradient is expressed by Equation 6.

∇J(s(u,v)|e(u,v))=−[e(u,v)−(|s(u,v)|²+|r(u,v)|²+s(u,v)r*(u,v)+s*(u,v)r(u,v))]×(s(u,v)+r(u,v)) [Equation 6]

A constraint or filter h(u, v) is added as a convolution, as expressed by Equation 7.

$${\hat{s} ( u , v )}_{new}^{n + 1} = h ( u , v ) * {\hat{s} ( u , v )}_{old}^{n + 1} \qquad [ Equation 7 ]$$

In Equation 7, the filter h(u, v) is similar to a low pass filter and a spread of the filter h(u, v) is limited by the estimated bandwidth B. At a subsequent iteration, ŝ(u,v)_{new}ⁿ⁺¹ is used as new estimate. A value of α is directly selected or estimated using a line-search algorithm. Once ŝ(u,v) is estimated, an image of specimen is obtained by back-propagating the complex function through convolution with a Fresnel impulse response as expressed by Equation 8.

$$i ( n , m ) = \frac{exp ( \frac{i 2 π }{λ} z )}{i λ z} {\sum }_{p = 0}^{P - 1} {\sum }_{q = 0}^{Q - 1} exp ( i \frac{π}{λ z} [ {( n Δ x - p Δ u )}^{2} + {( m Δ x - q Δ u )}^{2} ] ) \hat{s} ( p , q ) \qquad [ Equation 8 ]$$

In Equation 8, Δx and Δu denote sampling pixels in an imaged space and an inverse space of the imaged space. The sampling pixels are related by the magnification as expressed by Equation 9.

$$M = \frac{Δ u}{Δ x} = \frac{λ z}{{( N Δ x )}^{2}} \qquad [ Equation 9 ]$$

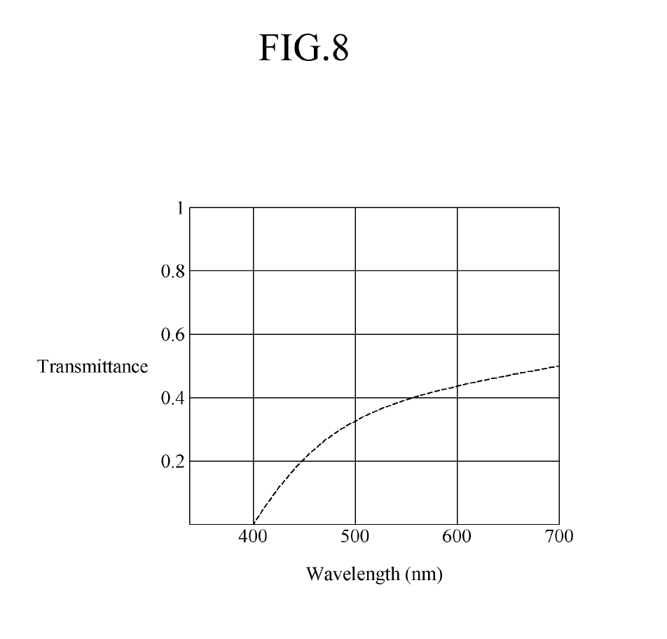

FIG. 8 illustrates an example waveform representing a relationship between light transmittance and a wavelength. Referring to the waveform of FIG. 8, when a wavelength of light generated from a light source is 550 nm, a transmittance is 0.4. When a wavelength of light generated from the light source is 600 nm, a transmittance increases to be greater than 0.4.

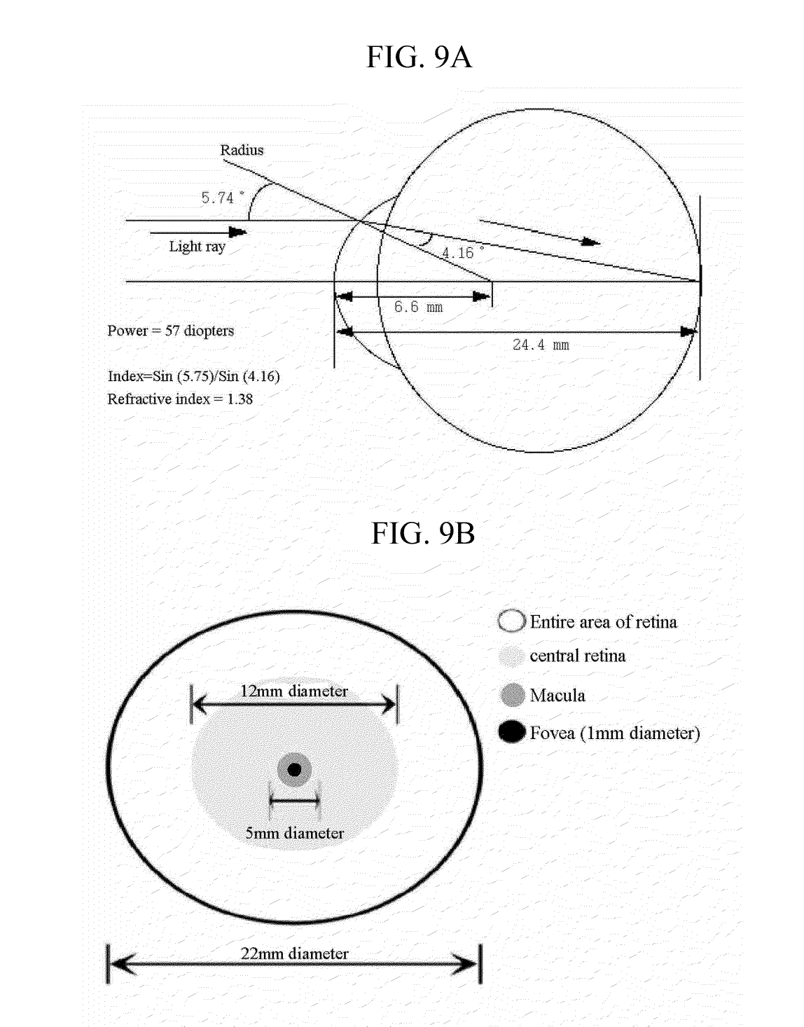

FIGS. 9A and 9B illustrate examples of a retinal dimension of an eye and a size of a donut-shaped illumination. The average size of the retina may be about 32 mm along a horizontal meridian of an eyeball, and the average area of the retina may be about 1094 mm². Incident light from a light source is to illuminate the entire area and an imaged area captured by an electronic device is to have a field-of-view (FOV). A refractive index of the eye at an average is estimated as 1.38. Since an imaging system is capable of having an effective numerical aperture of about 0.4 to 0.5, a central portion of the retina is easily imaged. Here, light is incident at 13° or less. Referring to FIG. 9A, the average distance between cornea and retina is 24.4 mm. As shown in FIG. 9B, the central retina has a diameter of 12 mm.

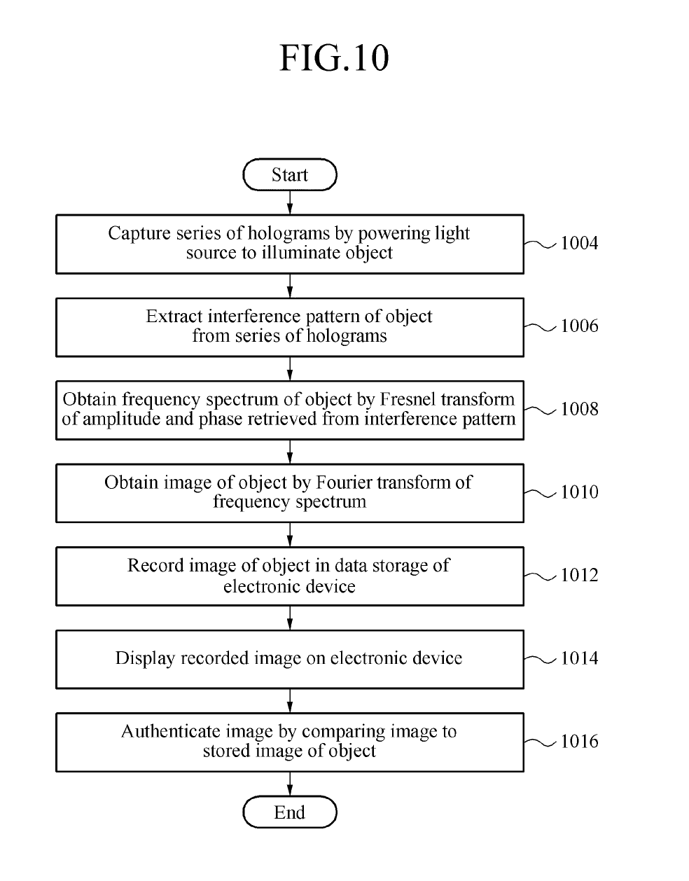

FIG. 10 is a flowchart illustrating a method of using the components disclosed in FIGS. 1 and 2 (e.g., the optical adapter 104 and the electronic device 106) to record an image of the object 102 of a user. The method includes capturing a series of holograms in operation 1004 by powering the light source 202 to illuminate the object 102 in operation 1004. The light from the light source 202 is collimated onto the object 102 through the pinhole aperture 204. The light source 202 is powered by triggering a button on the optical adapter 104 or a button on the electronic device 106. The controller 304 of FIG. 3 captures the series of holograms by powering the light source 202 to emit the light toward the object 102. For example, the proximal end 105a of the optical adapter 104 may be fixed to a smartphone, surrounding a camera on the smartphone. The distal end 105b of the optical adapter 104 is, for example, fixed to the skin of the user by the head strap 212.

In operation 1006, the method includes extracting an interference pattern of the object 102 from the series of holograms. The partially coherent light from the light source 202 is split by the beam-splitter cube 208 into an incident beam and a reference beam. The incident beam passes through the phase plate 206 and is reflected from a subject of the object 102. The incident beam is partially transmitted and partially reflected by the beam-splitter cube 208. The interference pattern is obtained by interference between the reflected beam from the object 102 and the reference beam. The controller 304 extracts the interference pattern of the object 102 from the series of holograms. Further, the controller 304 extracts the interference pattern prior to determining a calibration factor in the electronic device 106. For example, a camera of a smartphone may receive an interference pattern of the skin, an eye, or another object 102, by interference between an incident beam reflected back from the object 102 and a reference beam reflected back from the diffraction mirror 210.

In operation 1008, a frequency spectrum of the object 102 is obtained by obtaining a Fresnel transform of an amplitude and a phase retrieved from the interference pattern. More specifically, a high frequency portion in the frequency spectrum is recovered using an iterative restoration approach, and the controller 304 obtains a frequency spectrum of the object 102 by obtaining a Fresnel transform of an amplitude and a phase retrieved from the interference pattern. For example, the smartphone 106 may extract the frequency spectrum of the object 102 by obtaining the Fresnel transform of the amplitude and the phase recovered from the interference pattern.

In operation 1010, an image of the object 102 is obtained by obtaining a Fourier transform of the frequency spectrum. In an example, the image may be a low-resolution image. In another example, the image may be a high-resolution image. More specifically, when reconstructing the low-resolution image, the controller 304 obtains the image of the object 102 by obtaining the Fourier transform of the frequency spectrum. When reconstructing the high-resolution image, the controller 304 obtains the image of the object 102 by obtaining an inverse Fourier transform of the frequency spectrum. For example, the smartphone 106 reconstructs the image of the object 102 by the Fourier transform of the frequency spectrum when the image is a low-resolution image, and transmits the interference pattern to the server 108 for extracting spectrum data when the image is a high-resolution image.

In operation 1012, the reconstructed image of the object 102 is recorded in the data storage 310 of the electronic device 106. The data storage 310 records the image of the object 102 in the electronic device 106. For example, the smartphone 106 processes the interference pattern to record the image of the object 102 in a data storage 310 of the smartphone 106.

In operation 1014, the recorded image is displayed on the electronic device 106. The display screen 308 displays the recorded image on the electronic device 106. For example, the recorded image of the object 102 is displayed on the smartphone 106.

In operation 1016, the image is authenticated by comparing the recorded image to a pre-stored image of the object 102. The controller 304 authenticates the image by comparing the recorded image to the stored image of the object 102.

For example, an emergency room physician may use an optical adapter attached to an electronic device to view an eye of a user, for example, a patient. The optical adapter records images of the eye and transmits the images to the electronic device. The electronic device obtains a frequency spectrum of the eye by obtaining a Fresnel transform of an amplitude and a phase recovered from the captured image. The electronic device reconstructs the image of the eye by obtaining a Fourier transform of the frequency spectrum. The reconstructed image is stored in a data storage of the electronic device to diagnose the eye of the user, for example, the patient. Such a diagnosis is referred to as a coarse level diagnosis.

In another example, a medical practitioner may operate an imaging system while examining an eye of a user, for example, a patient to capture images of the eye. In this example, the captured images may be transmitted to an electronic device or a server to process and reconstruct an image of the eye and thereby diagnose the eye of the user, for example, the patient. Such a diagnosis is referred to as a detailed level diagnosis.

Various actions, acts, blocks, operations, and the like of FIG. 10 may be performed in order presented, in different order, or simultaneously. Further, in some examples, some actions, acts, blocks, operations, and the like may be omitted, added, modified, skipped, and the like without departing from the scope of the disclosure.

FIGS. 1 through 10 show an optical adapter that includes a separate light source and is attached to an electronic device including an imaging sensor, a controller, a communicator, a display screen, and a data storage to record an image of an object in order to diagnose existing or developing conditions. It is to be understood to a person having ordinary skill in the art that the examples may be achieved by an imaging system including the electronic device having the imaging sensor, the controller, the communicator, the display screen, and the data storage, and the optical adapter including its own light source and an optical system in the imaging system. It is also to be understood by a person of ordinary skill in the art that the examples may be achieved by the imaging system including components present in the optical adapter and components/modules present in the electronic device altogether without departing from the disclosure.

The examples disclosed herein may be implemented through at least one software program running on at least one hardware device and performing network management functions to control the elements.

The apparatuses, units, modules, devices, and other components illustrated in FIGS. 3 and 5 that perform the operations described herein with respect to FIGS. 6, 7 and 10 are implemented by hardware components. Examples of hardware components include controllers, sensors, generators, drivers, and any other electronic components known to one of ordinary skill in the art. In one example, the hardware components are implemented by one or more processors or computers. A processor or computer is implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices known to one of ordinary skill in the art that is capable of responding to and executing instructions in a defined manner to achieve a desired result. In one example, a processor or computer includes, or is connected to, one or more memories storing instructions or software that are executed by the processor or computer. Hardware components implemented by a processor or computer execute instructions or software, such as an operating system (OS) and one or more software applications that run on the OS, to perform the operations described herein with respect to FIGS. *. The hardware components also access, manipulate, process, create, and store data in response to execution of the instructions or software. For simplicity, the singular term “processor” or “computer” may be used in the description of the examples described herein, but in other examples multiple processors or computers are used, or a processor or computer includes multiple processing elements, or multiple types of processing elements, or both. In one example, a hardware component includes multiple processors, and in another example, a hardware component includes a processor and a controller. A hardware component has any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in FIGS. 6, 7 and 10 that perform the operations described herein with respect to FIGS. 3 and 5 are performed by a processor or a computer as described above executing instructions or software to perform the operations described herein.

Instructions or software to control a processor or computer to implement the hardware components and perform the methods as described above are written as computer programs, code segments, instructions or any combination thereof, for individually or collectively instructing or configuring the processor or computer to operate as a machine or special-purpose computer to perform the operations performed by the hardware components and the methods as described above. In one example, the instructions or software include machine code that is directly executed by the processor or computer, such as machine code produced by a compiler. In another example, the instructions or software include higher-level code that is executed by the processor or computer using an interpreter. Programmers of ordinary skill in the art can readily write the instructions or software based on the block diagrams and the flow charts illustrated in the drawings and the corresponding descriptions in the specification, which disclose algorithms for performing the operations performed by the hardware components and the methods as described above.

The instructions or software to control a processor or computer to implement the hardware components and perform the methods as described above, and any associated data, data files, and data structures, are recorded, stored, or fixed in or on one or more non-transitory computer-readable storage media. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, and any device known to one of ordinary skill in the art that is capable of storing the instructions or software and any associated data, data files, and data structures in a non-transitory manner and providing the instructions or software and any associated data, data files, and data structures to a processor or computer so that the processor or computer can execute the instructions. In one example, the instructions or software and any associated data, data files, and data structures are distributed over network-coupled computer systems so that the instructions and software and any associated data, data files, and data structures are stored, accessed, and executed in a distributed fashion by the processor or computer.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. An imaging system for using an optical device with an electronic device for diagnostic imaging, wherein the imaging system comprises:
a controller configured to
capture a series of holograms by powering a light source of the optical device to illuminate an object, wherein light from the light source is collimated onto the object through an aperture of the optical device,
extract an interference pattern of the object from the series of holograms, wherein the interference pattern is produced by interference between a reflected beam from the object and a reference beam formed by a diffraction mirror of the optical device, and
record at least one image of the object based on the interference pattern; and
a non-transitory data storage configured to store the at least one image.

2. The imaging system of claim 1, wherein, when recording the at least one image of the object based on the interference pattern, the imaging system is configured to:
obtain a frequency spectrum of the object by obtaining a Fresnel transform of an amplitude and a phase retrieved from the interference pattern, wherein a high frequency portion in the frequency spectrum is recovered using an iterative restoration approach; and
obtain the at least one image of the object by obtaining a Fourier transform of the frequency spectrum, wherein the at least one image is a low-resolution image.

3. The imaging system of claim 1, wherein, when recording the at least one image of the image based on the interference pattern, the imaging system is configured to:
obtain a frequency spectrum of the object by obtaining a Fresnel transform of an amplitude and a phase retrieved from the interference pattern, wherein a high frequency portion in the frequency spectrum is recovered using an iterative restoration approach; and
obtain the at least one image of the object by obtaining an inverse Fourier transform of the frequency spectrum, wherein the at least one image is a high-resolution image.

4. The imaging system of claim 1, wherein the light is partially reflected and partially transmitted by a beam splitter.

5. The imaging system of claim 4, wherein the light is split by the beam splitter into an incident beam and the reference beam, and the incident beam passes through a phase plate and is reflected from the object.

6. The imaging system of claim 1, wherein the reference beam is formed by the light source.

7. The imaging system of claim 1, wherein the optical device comprises an adaptor, the adaptor comprising:
a housing facility comprising a proximal end and a distal end, the housing facility being configured to removably attach to the electronic device at the proximal end, and
the proximal end is configured to surround an imaging sensor of the electronic device and the distal end is configured to be fixed on or near the object using a head strap.

8. The imaging system of claim 1, wherein the imaging system is further configured to:
display the recorded at least one image on the electronic device; and
authenticate the recorded at least one image by comparing the recorded at least one image to at least one pre-stored image of the object.

9. A method of operating an optical device, the method comprising:
capturing a series of holograms by powering a light source associated with the optical device to illuminate an object, wherein light from the light source is collimated onto the object through an aperture;
extracting an interference pattern of the object from the series of holograms, wherein the interference pattern is produced by interference between a reflected beam from the object and a reference beam formed by a diffraction mirror associated with the optical device;
recording at least one image of the object based on the interference pattern; and
storing the at least one image in a data storage of an electronic device.

10. The method of claim 9, wherein the recording of the at least one image comprises:
obtaining a frequency spectrum of the object by obtaining a Fresnel transform of an amplitude and a phase retrieved from the interference pattern, wherein a high frequency portion in the frequency spectrum is recovered using an iterative restoration approach; and
obtaining the at least one image of the object by obtaining a Fourier transform of the frequency spectrum, wherein the at least one image is a low-resolution image.

11. The method of claim 9, wherein the recording of the at least one image comprises:
obtaining a frequency spectrum of the object by obtaining a Fresnel transform of an amplitude and a phase retrieved from the interference pattern, wherein a high frequency portion in the frequency spectrum is recovered using an iterative restoration approach; and
obtaining the at least one image of the object by obtaining an inverse Fourier transform of the frequency spectrum, wherein the at least one image is a high-resolution image.

12. The method of claim 9, wherein the light is partially reflected and partially transmitted by a beam splitter.

13. The method of claim 12, wherein the light is split by the beam splitter into an incident beam and the reference beam, and the incident beam passes through a phase plate and is reflected from the object.

14. The method of claim 9, wherein the reference beam is formed by the light source.

15. The method of claim 9, further comprising:
displaying the recorded at least one image on the electronic device; and
authenticating the recorded at least one image by comparing the recorded at least one image to at least one pre-stored image of the object.

16. An imaging system for recording at least one image of an object, the imaging system comprising:
a housing facility comprising a light source, an aperture, a diffraction mirror, a head strap, a display screen, a data storage, and a controller, wherein:
the housing facility comprises a proximal end and a distal end, and is configured to attach to the display screen at the proximal end; and
the controller is configured to
capture a series of holograms by powering the light source to illuminate the object, wherein light from the light source is collimated onto the object through the aperture,
extract an interference pattern of the object from the series of holograms, wherein the interference pattern is produced by interference between a reflected beam from the object and a reference beam formed by the diffraction mirror,
record at least one image of the object based on the interference pattern, and
store the at least one image in the data storage.

17. The imaging system of claim 16, wherein, when recording the at least one image of the object based on the interference pattern, the controller is further configured to:
obtain a frequency spectrum of the object by obtaining a Fresnel transform of an amplitude and a phase retrieved from the interference pattern, wherein a high frequency portion in the frequency spectrum is recovered using an iterative restoration approach; and
obtain the at least one image of the object by obtaining a Fourier transform of the frequency spectrum, wherein the at least one image is a low-resolution image.

18. The imaging system of claim 16, wherein, when recording the at least one image of the object based on the interference pattern, the controller is further configured to:
obtain a frequency spectrum of the object by obtaining a Fresnel transform of an amplitude and a phase retrieved from the interference pattern, wherein a high frequency portion in the frequency spectrum is recovered using an iterative restoration approach; and
obtain the at least one image of the object by obtaining an inverse Fourier transform of the frequency spectrum, wherein the at least one image is a high-resolution image.

19. The imaging system of claim 18, wherein the light is partially reflected and partially transmitted by a beam splitter.

20. The imaging system of claim 16, wherein the controller is further configured to:
display the recorded at least one image on the display screen; and
authenticate the recorded at least one image by comparing the recorded at least one image to at least one pre-stored image of the object.

21. An imaging adaptor comprising:
a housing configured to attach to an image sensor of an electronic device, and configured to be fixed to or near an object, wherein the imaging adaptor is configured to
emit light towards the object,
capture a series of holograms generated by light reflected from the object, and
generate an interference pattern from the series of holograms, wherein the interference pattern is configured to be processed to record an image of the object.

22. The imaging adaptor of claim 21, wherein the electronic device is a smartphone.

23. The imaging adaptor of claim 21, wherein the object is an eye.
